(19) United States
(12) Patent Application Publication
(10) Pub. No.: US 2023/0372723 A1
(43) Pub. Date: Nov. 23, 2023

(54) DEFIBRILLATOR INTERFACE DEVICE FOR ATRIAL CARDIOVERSION THERAPY

(71) Applicant: NATIVE CARDIO, INC., Naples, FL (US)

(72) Inventors: Joseph PERGOLIZZI, Naples, FL (US); Diane LEVESQUE, Naples, FL (US); Gary Scott DIXON, Naples, FL (US); Young KIM, Naples, FL (US)

(21) Appl. No.: 18/030,629

(22) PCT Filed: Oct. 8, 2021
(86) PCT No.: PCT/US2021/054140

§ 371 Date: Apr. 6, 2023

Related U.S. Application Data
(60) Provisional application No. 63/089,287, filed on Oct. 8, 2020.

(51) Int. Cl. A61N 1/39

(52) U.S. Cl. CPC A61N 1/3956; A61N 1/3912

(57) ABSTRACT

Certain embodiments of the present disclosure relate to a defibrillator interface device for use in an atrial cardioversion system. In one embodiment, the defibrillator interface device comprises: an input terminal configured to receive an input signal from a defibrillator when coupled thereto; an output terminal configured to deliver an output signal comprising at least one discrete pulse to a plurality of heart lead wires when coupled thereto; and an attenuation circuit configured to generate an output signal by reducing the input signal energy.

### CROSS-REFERENCE TO RELATED APPLICATION(S)

This application claims the benefit of priority of U.S. Provisional Patent Application No. 63/089,287, filed on Oct. 8, 2020, the disclosure of which is hereby incorporated by reference herein in its entirety.

### TECHNICAL FIELD

This application relates to atrial cardioversion therapy and devices for use in the same.

### BACKGROUND

Cardiac rhythm disturbances, such as atrial fibrillation, are a common occurrence. Post-operative atrial fibrillation, in particular, is the most frequent heart rhythm disorder after cardiac surgery with its occurrence varying according to the underlying cardiac disease and procedure. Detection of post-operative atrial fibrillation is critical due to its frequency and because it is often associated with increased risk of death, nonfatal myocardial infarction, and nonfatal stroke.

Current practice usually dictates pharmacological treatments and/or electrical shock conversion for treatment of patients. Electrical shock conversion requires a high amount of energy and maintaining the sinus rhythm using this approach is difficult. Moreover, conventional approaches pose additional problems for treatment of patients suffering from abnormal cardiac rhythms. Conventional external electric defibrillation procedures typically require high electrical energy (i.e., on the order of 50-150 joules) and, as a result of the intense shock and patient discomfort associated therewith, are typically delivered after general anesthesia or deep sedation, both of which are time consuming procedures. In addition, the high amount of energy required to place a patient back into sinus rhythm requires costly drugs for sedation and can cause external skin damage and pain.

Moreover, external shock defibrillation is a time consuming process requiring additional medical personnel including anesthesiologists, cardiologists, and nurses. The procedure itself is not without substantial risk to the patient's life. The numerous complications, disadvantages, and failures associated with conventional systemic use of external electrical shock procedures has prompted many attempts to significantly improve treatment and patient care.

Although transthoracic electrical cardioversion is effective in sinus rhythm restoration, it is also associated with serious complications such as new arrhythmias, anesthesia-related challenges, and electrical skin burns in different tissue layers. To address this problem, attempts have been made to provide low-energy delivery through temporary epicardial leads as an alternative approach to the transthoracic electrical cardioversion. However, technical difficulties during the suturing on the atria, added time to the procedure, and the risk of bleeding during lead extraction make these approaches challenging.

### SUMMARY OF THE DISCLOSURE

The following summary presents a simplified summary of various aspects of the present disclosure in order to provide a basic understanding of such aspects. This summary is not an extensive overview of the disclosure. It is intended to neither identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In one aspect, a defibrillator interface device comprises: an input terminal configured to receive an input signal from a defibrillator when coupled thereto; an output terminal configured to deliver an output signal comprising at least one discrete pulse to a plurality of heart lead wires when coupled thereto; and an attenuation circuit configured to convert the input signal into the output signal such that a pulse energy for each discrete pulse in the output signal is less than 6 Joules.

In at least one embodiment, the pulse energy is less than 5.5 Joules, less than 5.0 Joules, less than 4.5 Joules, less than 4.0 Joules, less than 3.5 Joules, less than 3.0 Joules, less than 2.5 Joules, less than 2.0 Joules, less than 1.5 Joules, less than 1.0 Joules, less than 0.5 Joules, less than 0.4 Joules, less than 0.3 Joules, less than 0.25 Joules, or less than 0.2 Joules.

In at least one embodiment, at least one of the discrete pulses of the output signal comprises a square waveform, a plateau waveform, a truncated exponential waveform, a rounded waveform, or a monophasic or biphasic form thereof.

In at least one embodiment, the defibrillator interface device comprises a cardioversion cable coupled between the output terminal and the plurality of heart lead wires.

In at least one embodiment, the input terminal is configured to couple to the defibrillator via a cable connector without physical contact with defibrillator paddles.

In another aspect, a defibrillator interface device comprises: an input terminal configured to couple to a defibrillator; an output terminal configured to couple to a plurality of heart lead wires; and an attenuation circuit configured to couple the input terminal to the output terminal. In at least one embodiment, the attenuation circuit comprises a plurality of resistors arranged in a voltage divider configuration, wherein each of the plurality of resistors exhibits a resistance of less than 1 kΩ.

In at least one embodiment, the input terminal is coupled directly to the attenuation circuit without any intervening voltage discharge tubes.

In at least one embodiment, the resistors are selected such that a pulse energy of output pulses generated by the attenuation circuit is less than 6 Joules.

In at least one embodiment, the defibrillator interface device further comprises at least one voltage discharge tube coupled in parallel between the attenuation circuit and the output terminal.

In at least one embodiment, the input terminal is configured to couple to the defibrillator via a cable connector without physical contact with defibrillator paddles.

In another aspect, an atrial cardioversion system comprises: a defibrillator; a defibrillator interface device electrically coupled to the defibrillator via a cable connection without physical contact with defibrillator paddles; and a plurality of heart lead wires electrically coupled to the defibrillator interface device to deliver a signal from the defibrillator interface device to a patient's heart when coupled thereto.

In at least one embodiment, the defibrillator interface device is configured to convert an input pulse signal from the defibrillator into an output pulse signal that is transmitted to the patient's heart. In at least one embodiment, a pulse energy of the output pulse signal is less than 6 Joules.

In at least one embodiment, the pulse energy is less than 5.5 Joules, less than 5.0 Joules, less than 4.5 Joules, less than 4.0 Joules, less than 3.5 Joules, less than 3.0 Joules, less than 2.5 Joules, less than 2.0 Joules, less than 1.5 Joules, less than 1.0 Joules, less than 0.5 Joules, less than 0.4 Joules, less than 0.3 Joules, less than 0.25 Joules, or less than 0.2 Joules.

In at least one embodiment, the output pulse signal comprises a square waveform, a plateau waveform, a truncated exponential waveform, a rounded waveform, or a monophasic or biphasic form thereof.

In at least one embodiment, the system further comprises a cardioversion cable coupled between the defibrillator interface device and the plurality of heart lead wires.

In another aspect, a method of performing atrial cardioversion comprises: receiving, by an input terminal of a defibrillator interface device, an input pulse from a defibrillator via a cable connector coupling the defibrillator to the input terminal; converting, by an attenuation circuit, the input pulse into an output pulse having a pulse energy of less than 6 Joules; and transmitting the output pulse to a patient's heart via a plurality of heart lead wires in contact therewith.

In at least one embodiment, the pulse energy is less than 5.5 Joules, less than 5.0 Joules, less than 4.5 Joules, less than 4.0 Joules, less than 3.5 Joules, less than 3.0 Joules, less than 2.5 Joules, less than 2.0 Joules, less than 1.5 Joules, less than 1.0 Joules, less than 0.5 Joules, less than 0.4 Joules, less than 0.3 Joules, less than 0.25 Joules, or less than 0.2 Joules.

In at least one embodiment, the output pulse comprises a square waveform, a plateau waveform, a truncated exponential waveform, a rounded waveform, or a monophasic or biphasic form thereof.

In at least one embodiment, the method further comprises: coupling the input terminal of the defibrillator interface device via the cable connector without physically contacting the input terminal with defibrillator paddles.

In at least one embodiment, the attenuation circuit is electrically coupled to the input terminal without any intervening voltage discharge tubes.

In at least one embodiment, successful atrial cardioversion is achieved at defibrillation threshold of greater than 0.1 J and less than 1 J, less than 0.9 J, less than 0.8 J, less than 0.7 J, less than 0.6 J, less than 0.5 J, less than 0.4 J, less than 0.3 J, or less than 0.2 J. In at least one embodiment, the successful atrial cardioversion is achieved using single-stage energy delivery.

### BRIEF DESCRIPTION OF THE DRAWINGS

In order to facilitate a fuller understanding of the present disclosure, reference is now made to the accompanying drawings, in which like elements are referenced with like numerals. These drawings should not be construed as limiting the present disclosure, but are intended to be exemplary only.

FIG. 1 is a block diagram illustrating an exemplary atrial cardioversion system in accordance with certain embodiments.

FIG. 2 is a block diagram illustrating an exemplary defibrillator interface device in accordance with certain embodiments.

FIG. 3 is a schematic illustrating an attenuation circuit and gas discharge tubes in accordance with certain embodiments.

FIG. 4 shows an electrocardiogram tracing for induced atrial fibrillation.

FIG. 5 shows a complete reversion from induced atrial fibrillation to normal sinus rhythm utilizing a defibrillator interface device in accordance with certain embodiments.

FIG. 6 is a flow chart illustrating an exemplary method for performing cardioversion in accordance with certain embodiments.

### DETAILED DESCRIPTION

Embodiments of the present disclosure relate to a defibrillator interface device for use in an atrial cardio version system to reduce an amount of energy delivered by a defibrillator to a patient's heart, for example, during atrial cardioversion therapy. The defibrillator interface device includes an attenuation circuit to convert an input signal (received from the defibrillator) into an output signal with reduced energy (e.g., less than 6 Joules).

Post-operative atrial fibrillation is generally a benign clinical condition with a favorable prognosis, however, the complications that are associated with post-operative atrial fibrillation can increase patient mortality. Treatment objectives for post-operative atrial fibrillation include maintaining hemodynamic stability, controlling symptoms, and preventing thromboembolism. Guidelines for treatment are based on rate control or cardioversion achieved through pharmacotherapy or the use of electrical cardioversion, though there is no consensus for optimal medical therapy.

Electrical cardioversion treatment represents about 10% of treatment efforts for post-operative atrial fibrillation. This treatment method is known to produce side effects such as burns, arrhythmias, and pain, and can jeopardize anesthesia management. Furthermore, electrical cardioversion requires sedatives (e.g., midazolam, propofol, or ketamine), which are usually accompanied by adverse events.

The conventional approach in electrical cardioversion for treating atrial fibrillation uses initial energy of about 100 Joules (J), with increasing increments in 100 J if the initial shock fails up to a maximum of 400 J. The number of shocks that can be administered before labeling atrial fibrillation as refractory is debated, and there is no strong data that can assist in determining the number of shocks that can be safely delivered during external cardioversion. Other limitations with conventional approaches include risk of dislodgment of wires and increased risk of bleeding. In view of these limitations and others, it is desirable to develop alternative methods of energy delivery for atrial defibrillation cardioversion in both the chronic and post-operative settings.

The embodiments of the present disclosure address these and other limitations by providing a low-energy method for conversion from atrial fibrillation to normal sinus rhythm. The embodiments provide a safer, faster, and potentially painless alternative to the conventional electrical conversion approaches. Certain embodiments utilize a defibrillator interface device that can be used with commercially available defibrillators, pacemakers, and epicardial wires to advantageously and effectively achieve cardioversion using single-stage defibrillation energy threshold values of less than about 0.2 J. As used herein, “single-stage” in the context of energy delivery refers to energy delivery resulting from a single biphasic shock. Advantages of the embodiments described herein include, but are not limited to, successful low-energy cardioversion, reduced or minimal damage due to suturing or removal of leads at the atrial surface, and reducing the exposure to antiarrhythmic medications and their corresponding side effects.

In certain embodiments, connection to the defibrillator is achieved through a direct cable, which advantageously eliminates the need for defibrillator pads/paddles to make the electrical connection to the interface device. This also advantageously eliminates the need or use of a safety switch that is typically part of a defibrillator paddle or pad, as well as the need to ensure proper positioning of the paddles or pads. Elimination of the paddle/pad connection also removes the risks associated with the high-voltage and unprotected connections.

The design of current defibrillator interface devices utilize gas discharge tubes at their input terminals for each input. Gas discharge tubes act as open circuits until a threshold voltage is reached (i.e., spark-over voltage), at which point they conduct current. While they are typically used for input-output isolation and energy transfer indication in current systems, gas discharge tubes are responsible for several complications. For example, they block any input below the spark-over voltage, thus limiting the minimum energy transfer possible (generally to over 2 J). Gas discharge tubes also have associated voltage drops (typically 15V), resulting in losses in energy efficiency. Gas discharge tubes also distort the biphasic waveform of the output signal, and can potentially result in either of the two pulses of a biphasic pulse to fail to exceed the spark-over voltage. Further, gas discharge tubes at the input can also prevent the defibrillator from delivering a shock into the expected patient load due to load mismatch, resulting in failure to deliver the desired energy.

By avoiding the use of paddles/pads, this eliminates the need for input-output isolation, which allows for the elimination of gas discharge tubes at the input terminal. This advantageously avoids adverse impact on the shape of the output waveform, thus allowing for the delivery of well-defined pulses, such as square waveforms, plateau waveforms, truncated exponential waveforms, rounded waveforms, or monophasic or biphasic forms thereof, to the patient's heart.

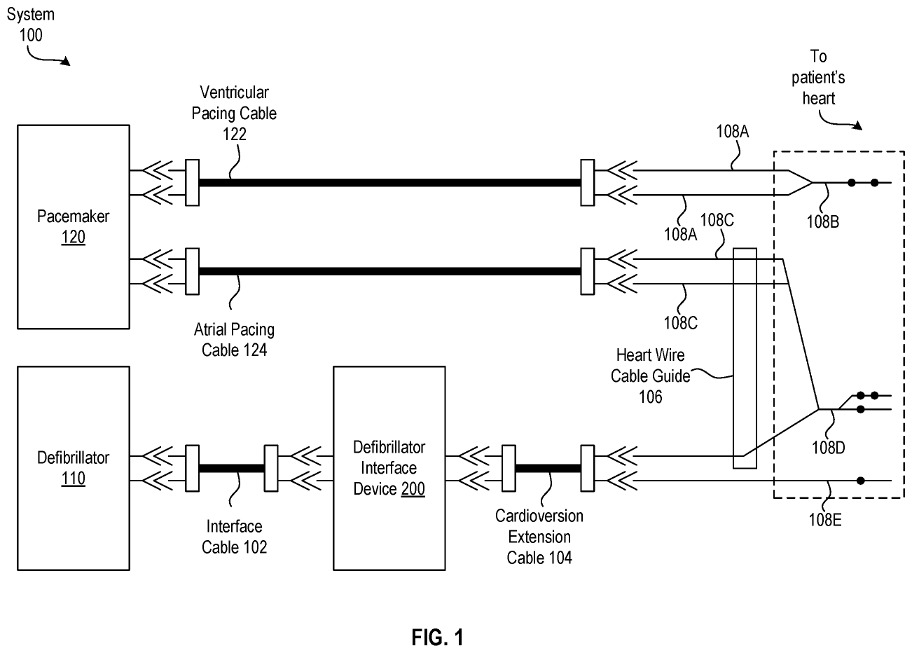

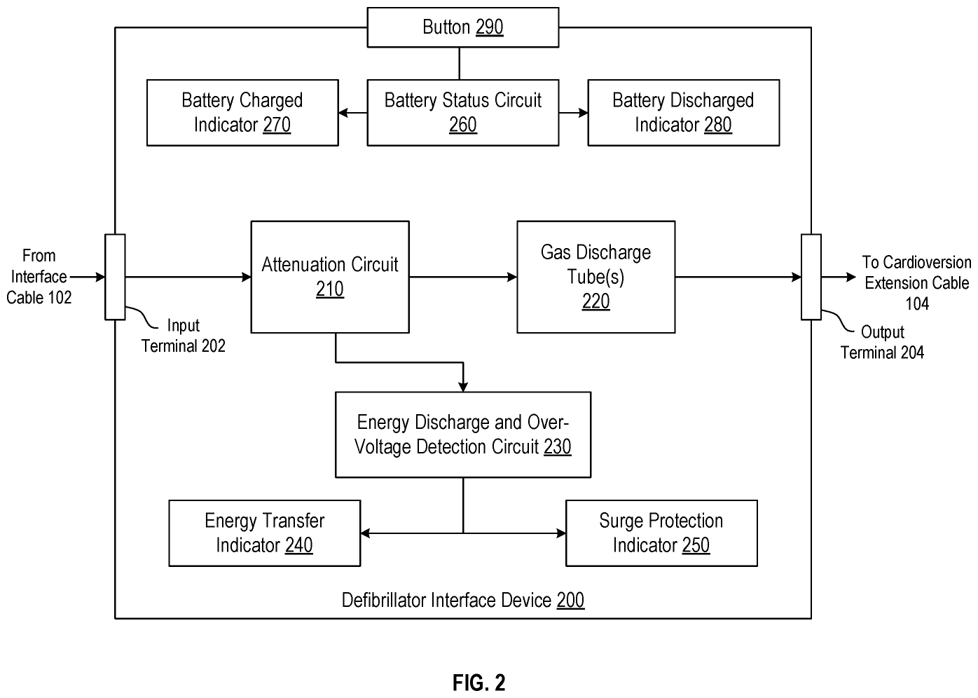

FIG. 1 is an atrial cardioversion system 100 in accordance with certain embodiments. The atrial cardioversion system 100 includes a defibrillator 110, a pacemaker 120, a defibrillator interface device 200, and a plurality of heart wires 108 adapted for interfacing with a patient's heart. The defibrillator interface device 200, which is discussed in greater detail with respect to FIG. 2, facilitates atrial cardioversion in cardiac surgery patients prone to post-operative atrial fibrillation. The heart wires 108, which include temporary pacing and cardioversion heart wires that are implanted during cardiac surgery, can be removed when the patient is no longer prone to post-operative atrial fibrillation, typically within seven days.

In certain embodiments, the defibrillator 110 is an external, adjustable, low-energy biphasic defibrillator that is capable of performing synchronized cardioversion (such as a LIFEPAK® defibrillator). The defibrillator 110 may be coupled to the defibrillator interface device 200 via an interface cable 102. The defibrillator interface device 200 is described in greater detail below with respect to FIG. 2.

The interface cable 102 may include a cable that is compatible with the defibrillator 110 (e.g., a LIFEPAK® QUIK-COMBO therapy cable). The interface cable 102 may directly connect the defibrillator 110 to an input terminal of the defibrillator interface device 200. In certain embodiments, the interface cable 102 is connected to the defibrillator interface device 200 without physically contacting the input terminal with defibrillator pads or paddles. For example, the interface cable 102 establishes a direct wired connection with the defibrillator interface device 200. In certain embodiments, the interface cable 102 may be produced by modifying a commercially-available cable that is compatible with the defibrillator 110 to remove the pads/paddles and connecting the exposed lead wires to the input terminal of the defibrillator interface device 200. In certain embodiments, to reduce stresses on the interface cable 102 at the defibrillator interface device 200, the input terminal of the defibrillator interface device 200 may include a strain relief connector that both seals and provides strain relief to the incoming lead wires of the interface cable 102. For example, the strain relief connector may include a tapered thread nut and/or a rubber o-ring.

The pacemaker 120 may be any suitable temporary external pacemaker compatible with atrial cardioversion, such as Medtronic models 5392 and 53401. In certain embodiments, the pacemaker 120 is capable of accepting two temporary bipolar pacing wires for ventricular and atrial pacing. The pacemaker 120 may connect to a subset of the heart wires 108 via a ventricular pacing cable 122 and an atrial pacing cable 124.

In certain embodiments, the ventricular pacing cable 122 is an extension cable that connects the pacemaker 120 to ventricular pacing wires 108A. The ventricular pacing cable 122 may be, for example, a Medtronic 5433V Reusable EPG ventricular safety cable or a Medtronic 5487 six-foot sterile disposable patient safety cable.

In certain embodiments, the atrial pacing cable 124 is an extension cable that connects the pacemaker 120 to atrial pacing heart wires 108C. The atrial pacing cable 124 may be, for example, a Medtronic 5433A Reusable EPG ventricular safety cable or a Medtronic 5487 six-foot sterile disposable patient safety cable.

In certain embodiments, a cardioversion extension cable 104 connects the defibrillator interface device 200 to at least a subset of the heart wires 108 to bridge the distance between the defibrillator interface device 200 and the patient's heart due to the short distance that the heart wires 108 extend from the exterior of the patient's body when implanted therein. The heart wires 108 provide direct connection to the patient's heart to enable cardiac pacing and atrial cardioversion. The heart wires 108 may be designed for implantation of up to seven days, and can be withdrawn with gentle traction.

In certain embodiments, the heart wires 108 include three different wires. The first wire is a ventricular wire 108B, which includes a bipolar lead that provides bipolar myocardial ventricular pacing. The lead may include a fixation mechanism distal to the electrodes. A proximal end of the lead may be bifurcated with a connector pin for each electrode.

The second wire is a right atrial wire 108D. The right atrial wire is a tripolar lead having a bifurcated distal end with one end providing a unipolar epicardial conversion electrode and the other end providing bipolar myocardial atrial pacing/sensing electrodes. The pacing fork end may include a fixation mechanism distal to the electrodes. The proximal end may be trifurcated with a connector pin for each electrode.

The third wire is a left atrial wire 108E, which is a unipolar epicardial cardio conversion lead.

Common features of the heart wires 108 include connectors, chest needles, and cardiac needles. In certain embodiments, the connectors are at proximal ends of the heart wires 108 and are unipolar pin-type connectors. Each connector in a given heart wire is attached the chest needle to allow the lead to be tunneled through the skin to be externalized. Exposed pins on pacing connects are covered when not connected to a pacing cable.

The most proximal end of heart wires 108 includes a straight chest needle attached to the connector pins. The straight chest needle may be used for tunneling the heart wire from its attachment to the heart for externalization through the skin. After externalization, it may be broken off of the connector pins, and the pins may be separated for connection to their respective devices.

It is to be understood that the configuration of the heart wires 108 and other components to couple the devices of the atrial cardioversion system 100 to the heart are merely illustrative, and other configurations may be utilized as would be appreciated by those of ordinary skill in the art. For example, in certain embodiments, the pacemaker 120 and associated components are omitted from the atrial cardioversion system 100. In such embodiments, the atrial cardioversion system 100 may utilize only utilize the defibrillator 110, the defibrillator interface device 200, and associated connectivity such that a pair of unipolar epicardial wires is used to connect the cardioversion extension cable 104 to the heart.

The most distal end of each heart wire branch may include a curved cardiac needle. The curved cardiac needle may be used to attach the electrodes to the heart. On pacing branches, the cardiac needle may also be used to embed a fixation mechanism in the myocardium for electrode placement fixation, and may be broken off after use.

The atrial cardioversion system 100 further utilizes a heart wire cable guide 106 to reduce stress where the right atrial wire 108D bifurcates to connect the atrial pacing heart wires 108C to the cardioversion extension cable 104.

FIG. 2 illustrates the defibrillator interface device 200 in accordance with certain embodiments. The defibrillator interface device 200 includes input terminal 202 (for connecting to the interface cable 102), an output terminal 204 (for connecting to the cardioversion extension cable 104), an attenuation circuit 210, and one or more gas discharge tubes 220. The defibrillator interface device 200 further includes an energy discharge and over-voltage detection circuit 230 (to indicate that the defibrillator has discharged a set energy amount, which is visually indicated by an energy transfer indicator 240), a surge protection indicator 250, a battery status circuit 260 with a battery charged indicator 270 and a battery discharged indicator 280 to visually represent the status of the battery, and a button 290 for checking the battery status. It is to be understood that some of the components may be omitted or modified, and that additional components may be present, as would be appreciated by those of ordinary skill in the art.

In certain embodiments, the attenuation circuit 210 is designed to attenuate the input signal from the defibrillator 110 to a level compatible with atrial cardioversion and to ensure that the defibrillator 110 sees a low impedance, for example, within the range of 52.3-57 ohms (Ω). In certain embodiments, the resistors of the attenuation circuit 210 are non-inductive so as to prevent or reduce phase shifts in the waveform to be delivered to the patient's heart. In certain embodiments, the attenuation circuit 210 may be designed to reduce an amount of energy per pulse to less than or equal to 6 Joules (J), less than or equal to than 5 J, less than or equal to 4 J, less than or equal to 3 J, less than or equal to 2 J, less than or equal to 1 J, less than or equal to 0.5 J, less than or equal to 0.2 J (such as about 0.17 J), less than or equal to 0.1 J, or within any range defined by any of these values (e.g., from 0.1 J to 2 J). Moreover, the attenuation circuit 210 in certain embodiments is coupled directly to the input terminal 202 without any intervening voltage discharge tubes.

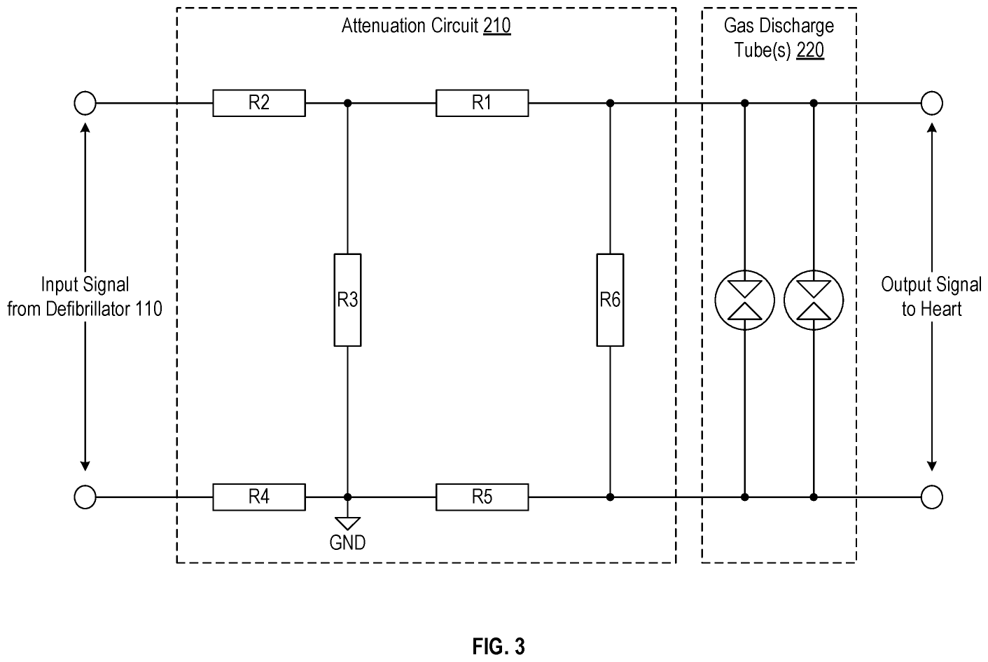

FIG. 3 is a schematic illustrating the attenuation circuit 210 in accordance with certain embodiments. In certain embodiments, the attenuation circuit 210 comprises a plurality of resistors arranged in a voltage divider configuration, wherein each of the plurality of resistors exhibits a resistance of less than 1 kΩ In certain embodiments, the resistors are selected such that: R1=20Ω, R2=17Ω, R3=20Ω, R4=20Ω, R5=20Ω, and R6=175Ω. The value for R6 may be selected to be less than 1.5 kΩ or less than 1 kΩ in order to be compatible with certain defibrillators, such as the LIFEPAK® 20 defibrillator. The value of R6, in certain embodiments, may be adjusted based on the output specifications of the defibrillator 110 to identify an impedance of the attenuation circuit 210 that is within a suitable range suitable for use (e.g., less than from about 180Ω to about 250Ω. By selecting R6 to be less than 1 kΩ (or specifically 175Ω) which results in a low defibrillator load resistance to about 40-43Ω, the voltage divider performance is not adversely affected. This is because most defibrillators tend to exhibit adaptive energy delivery by terminating the shock delivery when the selected energy is delivered (e.g., extending a pulse width to accommodate changes in output from the attenuation circuit 210.

In certain embodiments, R2 is selected to be lower than R1, R3, R4, and R5. For example, R2 may be selected to be 17Ω while R1, R3, and R4, and R5 are selected to be 20Ω. This lower resistance may be selected to achieve a target attenuation that is less than (e.g., about 80% of) the nominal patient load of 50Ω.

In certain embodiments, the defibrillator interface device 200 includes one or more gas discharge tubes 220 connected between the attenuation circuit 210 and the output terminal 204, which may be arranged in parallel as illustrated in FIG. 3. The one or more gas discharge tubes may be used to prevent an excessive shock voltage to be discharged to the patient. Gas discharge tubes maintain a high impedance off-state until a voltage exceeds a spark over voltage, at which point the gas in the gas discharge tube comes ionized resulting in a pulse of current that lasts less than a microsecond. During arc-over, the gas discharge tube exhibits low impedance resulting in very low on-state voltage (arc voltage). This effectively limits the over voltage to a low level and shunts the associated follow current away from downstream components and circuitry. When the surge event subsides and the system voltage returns to normal levels, the gas discharge tube will reset into its high impedance (off) state.

In certain embodiments, a spark over voltage rating of 400 V (or close to 400 V) is chosen for the gas discharge tubes to prevent discharges of 15 J or greater from being delivered to the patient's heart. In certain embodiments, multiple gas discharge tubes are used in the event that one of the gas discharge tubes fails to conduct at high voltages.

In certain embodiments, the energy discharge and-over voltage detection circuit 230 is separate from the attenuation circuit 210 and is designed to avoid interference with the attenuation circuit 210 and output signal. This can be achieved, for example, using a high impedance path tapped across resistor R3. In other embodiments, gas discharge tubes may be absent entirely from the defibrillator interface device 200.

In certain embodiments, the atrial cardioversion system 100 may be adapted to deliver one or more discrete pulses for treating atrial fibrillation or a pacing signal comprising a sequence of low energy pulses (having maximum current amplitudes of less than 0.1 milliamp) for treating atrial flutter. In certain embodiments, a discrete pulse is applied followed by application of a pacing signal. The energy E delivered to the heart is E(t)=V(t)I(t)t, Vis the output voltage of the defibrillator interface device 200, I(t) is the output current, and t is time. The amplitude of the output current I is related to the output voltage V by V=IR, where R is the resistance of the current flow path through the heart. The amount of energy for a given pulse can be computed by integrating over the output voltage V over the duration of the pulse. For a continuous sequence of pulses, the total energy delivered can be computed by integrating over the total length of time that the signal is applied to the heart. In certain embodiments, the voltage amplitude, duration, and shape of a discrete pulse may be selected so as to deliver a total amount of energy that is less than 6 Joules. For example, the duration of a discrete pulse may be 0.1 seconds or less Similarly, in certain embodiments, a pacing signal may be generated such that the current amplitude does not exceed 100 microamps (e.g., the maximum current amplitude remains less than or equal to 100 microamps, less than or equal to 50 microamps, less than or equal to 10 microamps, or less than or equal to 1 microamp), and the overall duration of the pacing signal is maintained such that the total energy delivery is less than 6 Joules.

### Illustrative Examples

The following examples are set forth to assist in understanding the disclosure and should not, of course, be construed as specifically limiting the embodiments described and claimed herein. Such variations of the embodiments, including the substitution of all equivalents now known or later developed, which would be within the purview of those skilled in the art, and changes in formulation or minor changes in experimental design, are to be considered to fall within the scope of the embodiments incorporated herein. All data are reported as means with standard deviations.

### Anesthesia and Surgical Procedure

Three male domestic swine weighing an average of 60.6±9 kg were used. The animals were pre-medicated with an intramuscular injection of Telazol (3 mg/kg). They were intubated under direct vision with an appropriately sized cuffed endotracheal tube, and anesthesia was performed with 1.5% isoflurane. Electrocardiography was continuously monitored using an electrophysiology record system (BARD Electrophysiology Lab System) throughout the procedure. The animals were placed in the dorsal recumbency position, prepped, and draped.

For each animal, the chest was opened via a median sternotomy and the heart was exposed. A pair of custom-made, stainless steel wire electrodes (Medtronic Streamline electrodes) each with 6 centimeters of a non-insulated area at the distal portion were weaved intra-myocardially one each into the right and left atrial appendages at the bases. The distal non-insulated portions were used as the cardioversion electrodes. Two to four bites were used while placing the leads to create a straight line. Sub-epicardial placement was preferred to decrease the risk of bleeding observed with full-thickness bites and to assist with tissue conductivity. All wires were extruded subcutaneously at the level of the subxiphoid process and secured with a non-absorbable suture.

### Impedance Measurement

Electrical impedance of the atrial tissue was measured using a known resistor of 100Ω. A constant 2V pulse signal with a pulse width of 0.5 milliseconds was sourced through a known resistor of 100Ω into the atrium through the atrial leads at a frequency of 1 hertz (Hz). The resulting voltage across the known resistor was measured using an oscilloscope. Atrial impedance was then calculated from these measured voltages.

### Atrial Fibrillation Induction

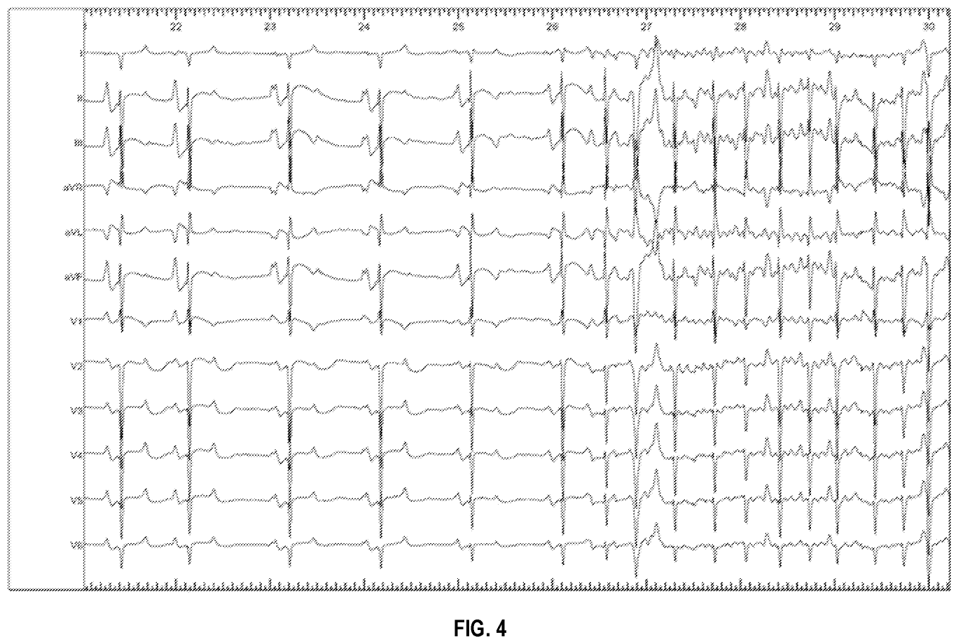

Atrial fibrillation was induced in a hybrid approach (pharmacological and electrical). A 2.5 mg Neostigmine IV bolus was given, followed by direct injection of 0.5 mg acetylcholine close to the SA node, at the junction of the right atrium and the superior vena cava (pharmacological approach). Subsequently, a 10 Hz pacing burst was delivered with wire electrodes or alligator clips on the surface of both atria (electrical approach) until atrial fibrillation was established. FIG. 4 shows the electrocardiogram tracing for atrial fibrillation induction, indicating the hybrid induction of atrial fibrillation with an injection of acetylcholine into the SVC/RA junction and burst pacing at 10 Hz. Electrocardiogram leads I, II, III, aVR, aVF, aVL, and V1-V6 were recorded during the entire procedure. Sustained atrial fibrillation was defined with the following criteria: one minute of the uninterrupted changes on the electrocardiogram such as lack of discrete ‘P’ waves, presence of ‘f’ waves, and a non-repetitive pattern of ventricular response.

### Atrial Fibrillation Cardioversion

Atrial cardioversion leads were connected to a defibrillator interface device as described herein (e.g., the defibrillator interface device 200), which served as a bridge to the defibrillator (LifePak 20, Medtronic). Although energy level selection was performed with the defibrillator, the defibrillator interface device attenuated and delivered the final output.

Ultra-low energy was delivered through the temporary atrial cardioversion leads sutured into the right and left atrium appendages as previously described. Shocks were synchronized to the ventricular R-wave to avoid inducing ventricular fibrillation. The initial shock energy was 0.15 J. If unsuccessful, the shock energy was increased in 0.03 J increments until 0.3 J, and then delivered at the following energy levels: 0.45 J, 0.6 J, 0.9 J, 1.5 J, 2.1 J, and 3 J. All energy levels were attempted at least twice before incrementing the energy to the next higher level.

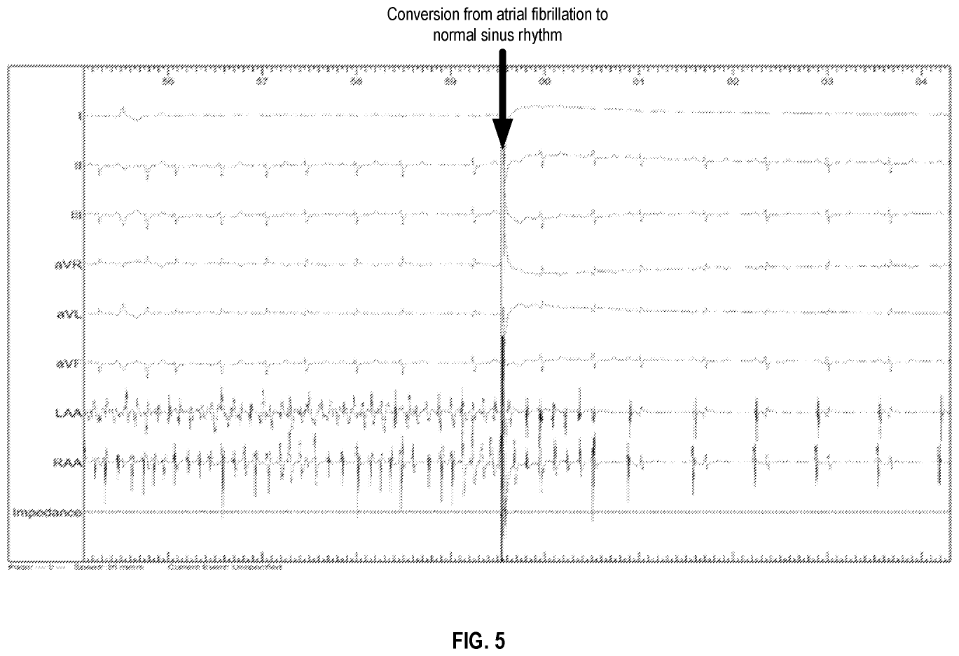

Successful electrical cardioversion was defined as a change from atrial fibrillation to normal sinus rhythm without early recurrence of atrial fibrillation within 60 seconds of cardioversion. A defibrillation threshold (DFT) was defined as the lowest shock energy required to successfully convert atrial fibrillation to normal sinus rhythm in two separate events. FIG. 5 shows a complete reversion from AFIB to sinus rhythm, at a DFT of 0.21 J.

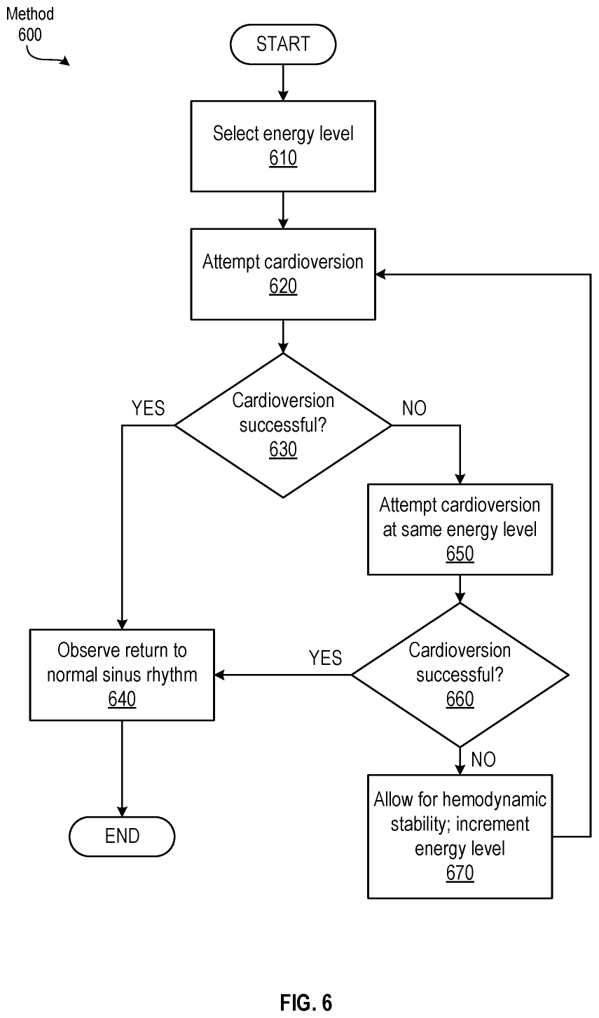

FIG. 6 is a flow chart illustrating an exemplary method 600 for performing cardioversion in accordance with certain embodiments, which summarizes the methodology described in this section. At block 610, an initial energy level is selected (e.g., 0.1 J, 0.15 J, 0.2 J, etc.). At block 620, cardioversion is attempted as described above. At block 630, if the cardioversion is successful, then the method 600 proceeds to block 640 where a return to normal sinus rhythm is observed. Otherwise, the method 600 proceeds to block 650. In certain embodiments, at block 650, cardioversion is attempted again at the same energy level. At block 660, if the cardioversion is successful, the method 600 proceeds to block 640. Otherwise, the method 600 proceeds to block 670, where time is allowed to elapse until hemodynamic stability is reached. The energy level is then incremented (e.g., 0.03 J increments, 0.3 J increments, etc.), and the method 600 proceeds to block 620. The attempts at cardioversion may be repeated until normal sinus rhythm is reached or until a maximum energy level is reached (e.g., 3 J).

### Leads Extraction

As discussed above, leads were attached to the epicardial surface of both right and left appendage in a straight-line shape and extruded subcutaneously at the level of the subxiphoid process. This technical detail was performed to mimic the placement of the temporary leads after cardiac surgery in the clinical setting. To assure the safety of leads removal, both wires were manually pulled back one following the other. Once the study was completed, the animals were euthanized under deep anesthesia and hearts underwent gross necropsy by the surgeon.

### Results

Atrial temporary lead placement was uneventful in all studies. A mean of 4.6±1.24 inductions of atrial fibrillation were performed, and the successful rate of induction was 100%. Table 1 summarizes the means values of energy, defibrillation threshold (DFT), and impedance for each study. No events of early recurrence of atrial fibrillation were noted during the duration of the studies. When the testing was concluded, temporary wires were removed with usual transcutaneous manual traction, the leads were easily removed, and no bleeding was observed. Gross necropsy did not exhibit any major injuries besides suturing bites. No shock-related injuries were observed.

The mean defibrillation threshold values in this study stand at 0.87±0.52 J, with the lowest DFT value being of 0.21 J, which are unexpectedly low for single-stage energy values for defibrillation with epicardial wires.

TABLE 1
Mean energy values used during the studies
and defibrillation thresholds

| Animal | Energy (Joules) | DFT (Joules) | | Impedance (ohms) | |
|---|---|---|---|---|---|
| 1 | 0.19 ± 0.05 J | 0.21 | J | 185 | Ω |
| 2 | 0.45 ± 0.39 J | 1.50 | J | 168 | Ω |
| 3 | 0.70 ± 0.14 J | 0.90 | J | 189 | Ω |
| Mean | 0.35 ± 0.30 J | 0.87 ± 0.52 | J | 181 ± 9 | Ω |

In the foregoing description, numerous details are set forth. It will be apparent, however, to one of ordinary skill in the art having the benefit of this disclosure, that the present disclosure may be practiced without these specific details. In some instances, well-known structures and devices are shown in block diagram form, rather than in detail, in order to avoid obscuring the present disclosure.

The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment” or “one embodiment” means that a particular feature, structure, or characteristic described in connection with the embodiment is included in at least one embodiment. Thus, the appearances of the phrase “an embodiment” or “one embodiment” in various places throughout this specification are not necessarily all referring to the same embodiment.

The present disclosure is not to be limited in scope by the specific embodiments described herein. Indeed, other various embodiments of and modifications to the present disclosure, in addition to those described herein, will be apparent to those of ordinary skill in the art from the description and accompanying drawings. Thus, such other embodiments and modifications are intended to fall within the scope of the present disclosure. Further, while the present disclosure has been described in the context of a particular embodiment in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein, along with the full scope of equivalents to which such claims are entitled.

1. A defibrillator interface device comprising:
an input terminal configured to receive an input signal from a defibrillator when coupled thereto;
an output terminal configured to deliver an output signal comprising at least one discrete pulse to a plurality of heart lead wires when coupled thereto; and
an attenuation circuit configured to convert the input signal into the output signal such that a pulse energy for each discrete pulse in the output signal is less than 6 Joules.

2. The defibrillator interface device of claim 1, wherein the pulse energy is less than 0.2 Joules.

3. The defibrillator interface device of claim 1, wherein at least one of the discrete pulses of the output signal comprises a square waveform, a plateau waveform, a truncated exponential waveform, a rounded waveform, or a monophasic or biphasic form thereof.

4. The defibrillator interface device of claim 1, further comprising a cardioversion cable coupled between the output terminal and the plurality of heart lead wires.

5. The defibrillator interface device of claim 1, wherein the input terminal is configured to couple to the defibrillator via a cable connector without physical contact with defibrillator paddles.

6. A defibrillator interface device comprising:
an input terminal configured to couple to a defibrillator;
an output terminal configured to couple to a plurality of heart lead wires; and
an attenuation circuit configured to couple the input terminal to the output terminal, wherein the attenuation circuit comprises a plurality of resistors arranged in a voltage divider configuration, wherein each of the plurality of resistors exhibits a resistance of less than 1 kΩ.

7. The defibrillator interface device of claim 6, wherein the input terminal is coupled directly to the attenuation circuit without any intervening voltage discharge tubes.

8. The defibrillator interface device of claim 6, wherein the resistors are selected such that a pulse energy of output pulses generated by the attenuation circuit is less than 6 Joules.

9. The defibrillator interface device of claim 6, further comprising at least one voltage discharge tube coupled in parallel between the attenuation circuit and the output terminal.

10. The defibrillator interface device of claim 6, wherein the input terminal is configured to couple to the defibrillator via a cable connector without physical contact with defibrillator paddles.

11-15. (canceled)

16. A method of performing atrial cardioversion, the method comprising:
receiving, by an input terminal of a defibrillator interface device, an input pulse from a defibrillator via a cable connector coupling the defibrillator to the input terminal;
converting, by an attenuation circuit, the input pulse into an output pulse having a pulse energy of less than 6 Joules; and
transmitting the output pulse to a patient's heart via a plurality of heart lead wires in contact therewith.

17. The method of claim 16, wherein the pulse energy is less than 0.2 Joules.

18. The method of claim 16, wherein the output pulse comprises a square waveform, a plateau waveform, a truncated exponential waveform, a rounded waveform, or a monophasic or biphasic form thereof.

19. The method of any of claim 16, further comprising:
coupling the input terminal of the defibrillator interface device via the cable connector without physically contacting the input terminal with defibrillator paddles.

20. The method of claim 16, wherein the attenuation circuit is electrically coupled to the input terminal without any intervening voltage discharge tubes.

21. The method of claim 16, wherein successful atrial cardioversion is achieved at defibrillation threshold of greater than 0.1 J and less than 1 J, less than 0.9 J, less than 0.8 J, less than 0.7 J, less than 0.6 J, less than 0.5 J, less than 0.4 J, less than 0.3 J, or less than 0.2 J.

22. The method of claim 21, wherein the successful atrial cardioversion is achieved using single-stage energy delivery.
